(19) United States
(12) Patent Application Publication
(10) Pub. No.: US 2013/0004498 A1
(43) Pub. Date: Jan. 3, 2013

(54) DOSING FOR TREATMENT WITH ANTI-EGFL7 ANTIBODIES

(75) Inventors: Shuang Bai, Sunnyvale, CA (US); Daniel S. Chen, Burlingame, CA (US); Roel Funke, Brisbane, CA (US); Priti Hegde, San Mateo, CA (US); Louie Naumovski, Redwood City, CA (US)

(73) Assignee: Genentech, Inc., South San Francisco, CA (US)

(21) Appl. No.: 13/364,135

(22) Filed: Feb. 1, 2012

Related U.S. Application Data
(60) Provisional application No. 61/438,944, filed on Feb. 2, 2011.
(60) Provisional application No. 61/492,743, filed on Jun. 2, 2011.
(60) Provisional application No. 61/587,382, filed on Jan. 17, 2012.

(51) Int. Cl. A61K 39/395; A61P 35/00; A61P 35/02; A61P 9/00

(52) U.S. Cl. 424/136.1; 424/158.1; 424/133.1

(57) ABSTRACT

The present invention concerns dosing of anti-EGFL7 antibodies for cancer therapy.

### PRIORITY OF INVENTION

This application claims priority under 35 U.S.C. §119 (e) to U.S. Provisional No. 61/438,944, filed Feb. 2, 2011; U.S. Provisional No. 61/492,743, filed Jun. 2, 2011; and U.S. Provisional No. 61/587,382, filed Jan. 17, 2012.

### FIELD OF THE INVENTION

The present invention relates to the treatment of cancer. More specifically, the invention concerns specific treatment of human patients susceptible to or diagnosed with cancer using anti-EGFL7 antibodies.

### BACKGROUND OF THE INVENTION

It is now well established that angiogenesis, which involves the formation of new blood vessels from preexisting endothelium, is implicated in the pathogenesis of a variety of disorders. These include solid tumors and metastasis, atherosclerosis, retrolental fibroplasia, hemangiomas, chronic inflammation, intraocular neovascular syndromes such as proliferative retinopathies, e.g., diabetic retinopathy, age-related macular degeneration (AMD), neovascular glaucoma, immune rejection of transplanted corneal tissue and other tissues, rheumatoid arthritis, and psoriasis. Folkman et al., J. Biol. Chem., 267: 10931-10934 (1992); Klagsbrun et al., Annu. Rev. Physiol., 53: 217-239 (1991); and Garner A., “Vascular diseases”, In: Pathobiology of Ocular Disease. A Dynamic Approach, Garner A., Klintworth GK, eds., 2nd Edition (Marcel Dekker, NY, 1994), pp 1625-1710. In the case of tumor growth, angiogenesis appears to be crucial for the transition from hyperplasia to neoplasia, and for providing nourishment for the growth and metastasis of the tumor. Folkman et al., Nature, 339: 58 (1989). The neovascularization allows the tumor cells to acquire a growth advantage and proliferative autonomy compared to normal cells.

The process of vascular development is tightly regulated. To date, a significant number of molecules, mostly secreted factors produced by surrounding cells, have been shown to regulate EC differentiation, proliferation, migration and coalescence into cord-like structures. For example, vascular endothelial growth factor (VEGF) has been identified as the key factor involved in stimulating angiogenesis and in inducing vascular permeability. Ferrara et al., Endocr. Rev., 18: 4-25 (1997). The role of other molecules in this process is also being elucidated, including Epidermal growth factor-like domain 7 (EGFL7; see, e.g., WO 2005/117968, filed Apr. 14, 2005). Inhibition of EGFL7 has been shown to be effective at inhibiting angiogenesis and treating tumors (see, e.g., WO 2007/106915, filed Mar. 16, 2007, and US-2010-0285009-A1, filed May 7, 2010).

In view of the role of vasculogenesis and angiogenesis in many diseases and disorders and the roled of EGFL7 in this process, it is desirable to have a means of reducing or inhibiting one or more of the biological effects causing these processes. All references cited herein, including patent applications and publications, are incorporated by reference in their entirety.

### SUMMARY OF THE INVENTION

Targeting EGFL7 presents an important and advantageous therapeutic modality for the treatment of cancer, especially in combination with other anti-angiogenic agents, e.g. anti-VEGF agents. The invention is in part based on the identification of the most efficacious dose of anti-EGFL7 antibodies for treating cancer and the surprising discovery that the optimal dose is significantly below the maximum tolerated dose. Accordingly, the invention provides methods, compositions, kits and articles of manufacture related to same.

For example, in some embodiments, the invention provides a method for the treatment of cancer in a human patient comprising administering an anti-EGFL7 antibody, the method comprising administering the antibody at a dose of between 1 mg/kg and 15 mg/kg. In some embodiments, the dose is between about 5 mg/kg and about 7.5 mg/kg. In some embodiments, the dose is about 5 mg/kg. In some embodiments, the dose is about 7.5 mg/kg.

In some embodiments, the invention provides a method for the treatment of cancer in a human patient comprising administering an anti-EGFL7 antibody, the method comprising administering the antibody at a flat dose selected from the group consisting of: (a) 375-400 mg every two weeks and (b) 550-600 mg every three weeks. In some embodiments, the flat dose is 375-400 mg every two weeks. In some embodiments, the flat dose is 550-600 mg every three weeks. In some embodiments the flat dose is 400 mg every two weeks. In some embodiments the flat dose is 600 mg every three weeks.

In some embodiments, the invention provides a method for the treatment of cancer in a human patient comprising administering an anti-EGFL7 antibody, the method comprising administering a first dose of the anti-EGFL7 antibody to the patient and a second dose of the anti-EGFL7 antibody to the patient, wherein the first dose and the second dose are each between 1 mg/kg and 15 mg/kg and the second dose follows the first does by between 1 and 4 weeks. In some embodiments, the first dose and the second dose are each between 5 mg/kg and 7.5 mg/kg and the second dose follows the first dose by between 2 and 3 weeks. In some embodiments, the first dose and the second dose are each 5 mg/kg and the second dose follows the first dose by 2 weeks. In some embodiments, the first dose and the second dose are each 7.5 mg/kg and the second dose follows the first dose by 3 weeks.

In some embodiments, the anti-EGFL7 antibody comprises a variable domain comprising to the following HVR sequences: (i) HVR-L1 comprising KASQSVDYSGDSYMS (SEQ ID NO: 1); (ii) HVR-L2 comprising GASYRES (SEQ ID NO: 2); (iii) HVR-L3 comprising QQNNEEPYT (SEQ ID NO: 3); (iv) HVR-H1 comprising GHTFTTYGMS (SEQ ID NO: 4); (v) HVR-H2 comprising GWINTHSGVPTYADDFKG (SEQ ID NO: 5); and (vi) HVR-H3 comprising LGSYAVDY (SEQ ID NO: 6). In some embodiments, the anti-EGFL7 antibody comprises the following heavy chain variable region sequence: EVQLVESGGGLVQPGGSLRLSCAASGHTFTTYGMSWVRQAPGKGLEWVGWINTHSGVP TYADDFKGRFTISLDNSKNTAYLQMNSLRAEDTAVYYCARLGSYAVDYWGQGTLVTVS S (SEQ ID NO: 7). In some embodiments, the anti-EGFL7 antibody comprises the following heavy chain variable region sequence: EIQLVESGGGLVQPGGSLRLSCAASGHTFTTYGMSWVRQAPGKGLEWMGWINTHSGVP TYADDFKGRFTISLDNSKSTAYLQMNSLRAEDTAVYFCARLGSYAVDYWGQGTLVTVS S (SEQ ID NO: 8). In some embodiments, the anti-EGFL7 antibody comprises the following light chain variable region sequence:

(SEQ ID NO: 9)
DIQMTQSPSSLSASVGDRVTITCKASQSVDYSGDSYMSWYQQKPGKAP
KLLIYGASYRESGVPSRFSGSGSGTDFTLTISSLQPEDFATYYCQQNN
EEPYTFGQGTKVEIKR.

In some embodiments, the anti-EGFL7 antibody comprises a variable domain comprising the following HVR sequences: (i) HVR-L1 comprising RTSQSLVHINGITYLH (SEQ ID NO: 10); (ii) HVR-L2 comprising RVSNRFS (SEQ ID NO: 11); (iii) HVR-L3 comprising GQSTHVPLT (SEQ ID NO: 12); (iv) HVR-H1 comprising GYTFIDYYMN (SEQ ID NO: 13); (v) HVR-H2 comprising GDINLDNGGTHYNQKFKG (SEQ ID NO: 14); and (vi) HVR-H3 comprising AREGVYHDYDDYAMDY (SEQ ID NO: 15). In some embodiments, the anti-EGFL7 antibody comprises the following heavy chain variable region sequence: EVQLVESGGGLVQPGGSLRLSCAASGYTFIDYYMNWVRQAPGKGLEWVGDINLDNGGT HYNQKFKGRFTISRDKSKNTAYLQMNSLRAEDTAVYYCAREGVYHDYDDYAMDWG QGTLVTVSS (SEQ ID NO: 16). In some embodiments, the anti-EGFL7 antibody comprises the following light chain variable region sequence:

(SEQ ID NO: 17)
DIQMTQSPSSLSASVGDRVTITCRTSQSLVHINGITYLHWYQQKPGKA
PKLLIYRVSNRFSGVPSRFSGSGSGTDFTLTISSLQPEDFATYYCGQS
THVPLTFGQGTKVEIKR.

In some embodiments, the anti-EGFL7 antibody comprises a variable domain comprising to the following HVR sequences: (i) HVR-L1 comprising RTSQSLVHINAITYLH (SEQ ID NO: 18); (ii) HVR-L2 comprising RVSNRFS (SEQ ID NO: 11); (iii) HVR-L3 comprising GQSTHVPLT (SEQ ID NO: 12); (iv) HVR-H1 comprising GYTFIDYYMN (SEQ ID NO: 13); (v) HVR-H2 comprising GDINLDNSGTHYNQKFKG (SEQ ID NO: 19); and (vi) HVR-H3 comprising AREGVYHDYDDYAMDY (SEQ ID NO: 15). In some embodiments, the anti-EGFL7 antibody comprises the following heavy chain variable region sequence: EVQLVESGGGLVQPGGSLRLSCAASGYTFIDYYMNWVRQAPGKGLEWVGDINLDNSGT HYNQKFKGRFTISRDKSKNTAYLQMNSLRAEDTAVYYCAREGVYHDYDDYAMDYVVG QGTLVTVSS (SEQ ID NO: 20). In some embodiments, the anti-EGFL7 antibody comprises the following light chain variable region sequence:

(SEQ ID NO: 21)
DIQMTQSPSSLSASVGDRVTITCRTSQSLVHINAITYLHWYQQKPGKA
PKWYRVSNRFSGVPSRFSGSGSGTDFTLTISSLQPEDFATYYCGQSTH
VPLTFGQGTKVEIKR.

In some embodiments, the antibody is administered in an infusion of 10, 20 or 30 minutes.

In some embodiments, the method further comprises the step of administering another anti-angiogenic agent. In some embodiments, the other anti-angiogenic agent is an anti-vascular endothelial growth factor (VEGF) antagonist. In some embodiments, the anti-VEGF antagonist is an anti-VEGF antibody. In some embodiments, the anti-VEGF antibody is bevacizumab.

In some embodiments, the anti-EGFL7 antibody is a bispecific antibody. In some embodiments, the bispecific antibody binds to VEGF. In some embodiments, the bispecific antibody binds to the same VEGF epitope as bevacizumab.

In some embodiments, the cancer is selected from group consisting of breast cancer, leukemia, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colon cancer, colorectal cancer, endometrial carcinoma, salivary gland carcinoma, kidney cancer, liver cancer, prostate cancer, vulvar cancer, thyroid cancer, hepatic carcinoma and various types of head and neck cancer. In some embodiments, the cancer is breast cancer, NSCLC or CRC.

In some embodiments, the method further comprises administering an effective amount of a chemotherapeutic agent.

The invention provides a method for the treatment of NSCLC in a human patient, comprising a dosing regimen comprising treatment cycles, wherein the patient is administered, on day 1 of each cycle, 200 mg/m2 pactlitaxel, carboplatin (AUC of 6 mg/ml min), 15 mg/kg bevacizumab, and 600 mg of an anti-EGFL7 antibody, each cycle being repeated every 21 days. In some embodiments, the paclitaxel and carboplatin are administered until disease progression of for up to 6 cycles. In some embodiments, the bevacizumab and anti-EGFL7 antibody are administered until disease progression or for up to 34 cycles. The anti-EGFL7 antibody may be any one of the anti-EGFL7 antibodies described herein.

The invention also provides a method for the treatment of colorectal cancer in a human patient, comprising a dosing regimen comprising treatment cycles, wherein the patient is administered, on day 1 of the first cycle, 85 mg/m2 oxaliplatin, 400 mg/m25-fluorourcail (5-FU), 400 mg/m2 folinic acid, 5 mg/kg bevacizumab, and 400 mg of an anti-EGFL7 antibody, and wherein the patient is administered on day 1 of each subsequent cycle, 85 mg/m2 oxaliplatin, 2400 mg/m2 5-FU, 400 mg/m2 folinic acid, 5 mg/kg bevacizumab, and 400 mg of an anti-EGFL7 antibody, each cycle being repeated every 14 days. In some embodiments, the oxaliplatin is administered for up to 8 cycles. In some embodiments, the 5-FU, folinic acid, bevacizumab and anti-EGFL7 antibody are administered until disease progression or for up to 52 cycles. The anti-EGFL7 antibody may be any one of the anti-EGFL7 antibodies described herein.

In some embodiments, the invention provides an article of manufacture comprising a container, a composition within the container comprising an anti-EGFL7 antibody, and a label or package insert with instructions to administer the antibody at a dose of between 1 mg/kg and 15 mg/kg.

In some embodiments, the invention provides an article of manufacture comprising a container, a composition within the container comprising an anti-EGFL7 antibody, and a label or package insert with instructions to administer a first dose of an anti-EGFL7 antibody to the patient and a second dose of an anti-EGFL7 antibody to the patient, wherein the first dose and the second dose are each between 1 mg/kg and 15 mg/kg and the second dose follows the first does by between 1 and 4 weeks. In some embodiments, the first dose and the second dose are each between 5 mg/kg and 7.5 mg/kg and the second dose follows the first dose by between 2 and 3 weeks. In some embodiments, the first dose and the second dose are each 5 mg/kg and the second dose follows the first dose by 2 weeks. In some embodiments, the first dose and the second dose are each 7.5 mg/kg and the second dose follows the first dose by 3 weeks.

In some embodiments, the invention provides an article of manufacture comprising a container, a composition within the container comprising an anti-EGFL7 antibody, and a label or package insert with instructions to administer the antibody at a flat dose selected from the group consisting of: (a) 375-400 mg every two weeks and (b) 550-600 mg every three weeks. In some embodiments, the flat dose is 375-400 mg every two weeks. In some embodiments, the flat dose is 550-600 mg every three weeks. In some embodiments the flat dose is 400 mg every two weeks. In some embodiments the flat dose is 600 mg every three weeks.

### BRIEF DESCRIPTION OF THE FIGURES

FIG. 1 depicts the design of Phase Ia and Phase Ib clinical trials.

FIG. 2 depicts PD biomarkers (CPCs) from Phase Ia.

FIG. 3 depicts PD biomarkers (CPCs) from Phase Ib.

### DETAILED DESCRIPTION OF THE INVENTION

The invention provides methods, compositions, kits and articles of manufacture relating to anti-EGFL7 antibodies and the treatment of cancer.

Details of these methods, compositions, kits and articles of manufacture are provided herein.

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### DEFINITIONS

The term “EGFL7” (interchangeably termed “Epidermal growth factor-like domain 7”), as used herein, refers, unless specifically or contextually indicated otherwise, to any native or variant (whether native or synthetic) EGFL7 polypeptide. The term “native sequence” specifically encompasses naturally occurring truncated or secreted forms (e.g., an extracellular domain sequence), naturally occurring variant forms (e.g., alternatively spliced forms) and naturally-occurring allelic variants.

The term “anti-EGFL7 antibody” or “an antibody that binds to EGFL7” refers to an antibody that is capable of binding EGFL7 with sufficient affinity such that the antibody is useful as a diagnostic and/or therapeutic agent in targeting EGFL7. In certain embodiments, an antibody that binds to EGFL7 has a dissociation constant (Kd) of <1 μM, <100 nM, <10 nM, <1 nM, or <0.1 nM. Anti-EGFL7 antibodies specifically include those described in WO 2007/106915, filed Mar. 16, 2007, and US-2010-0285009-A1, filed May 7, 2010.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

In one embodiment, the “Kd” or “Kd value” according to this invention is measured by a radiolabeled antigen binding assay (RIA) performed with the Fab version of an antibody of interest and its antigen as described by the following assay that measures solution binding affinity of Fabs for antigen by equilibrating Fab with a minimal concentration of (¹²⁵I)-labeled antigen in the presence of a titration series of unlabeled antigen, then capturing bound antigen with an anti-Fab antibody-coated plate (Chen, et al., (1999) J. Mol. Biol 293:865-881). To establish conditions for the assay, microtiter plates (Dynex) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab of interest (e.g., consistent with assessment of an anti-VEGF antibody, Fab-12, in Presta et al., (1997) Cancer Res. 57:4593-4599). The Fab of interest is then incubated overnight; however, the incubation may continue for a longer period (e.g., 65 hours) to insure that equilibrium is reached. Thereafter, the mixtures are transferred to the capture plate for incubation at room temperature (e.g., for one hour). The solution is then removed and the plate washed eight times with 0.1% Tween™-20 in PBS. When the plates have dried, 150 μl/well of scintillant (MicroScint™-20; Packard) is added, and the plates are counted on a TopCount gamma counter (Packard) for ten minutes. Concentrations of each Fab that give less than or equal to 20% of maximal binding are chosen for use in competitive binding assays. According to another embodiment the Kd or Kd value is measured by using surface plasmon resonance assays using a BIAcore™-2000 or a BIAcore™-3000 (BIAcore, Inc., Piscataway, N.J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 μg/ml (0.2 μM) before injection at a flow rate of 5 μl/minute to achieve approximately 10 response units (RU) of coupled protein. Following the injection of antigen, 1M ethanolamine is injected to block unreacted groups. For kinetics measurements, two-fold serial dilutions of Fab (0.78 nM to 500 nM) are injected in PBS with 0.05% Tween™ 20 (PBST) at 25° C. at a flow rate of approximately 25 μl/min. Association rates (kₒₙ) and dissociation rates (k_{off}) are calculated using a simple one-to-one Langmuir binding model (BIAcore™ Evaluation Software version 3.2) by simultaneous fitting the association and dissociation sensorgram. The equilibrium dissociation constant (Kd) is calculated as the ratio k_{off}/kₒₙ. See, e.g., Chen, Y., et al., (1999) J. Mol. Biol. 293:865-881. If the on-rate exceeds 10⁶ M⁻¹ S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM Aminco spectrophotometer (ThermoSpectronic) with a stirred cuvette.

The terms “antibody” and “immunoglobulin” are used interchangeably in the broadest sense and include monoclonal antibodies (e.g., full length or intact monoclonal antibodies), polyclonal antibodies, multivalent antibodies, multispecific antibodies (e.g., bispecific antibodies so long as they exhibit the desired biological activity) and may also include certain antibody fragments (as described in greater detail herein). An antibody can be human, humanized and/or affinity matured.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions or hypervariable regions (CDRs or HVRs, used interchangeably herein) both in the light-chain and the heavy-chain variable domains. The more highly conserved portions of variable domains are called the framework (FR). The variable domains of native heavy and light chains each comprise four FR regions, largely adopting a β-sheet configuration, connected by three HVRs, which form loops connecting, and in some cases forming part of, the β-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG₁, IgG₂, IgG₃, IgG₄, IgA₁, and IgA₂. The heavy-chain constant domains that correspond to the different classes of immunoglobulins are called α, δ, ε, γ, and μ, respectively. The subunit structures and three-dimensional configurations of different classes of immunoglobulins are well known.

“Antibody fragments” comprise only a portion of an intact antibody, wherein the portion preferably retains at least one, preferably most or all, of the functions normally associated with that portion when present in an intact antibody. Examples of antibody fragments include Fab, Fab′, F(ab′)2, and Fv fragments; diabodies; linear antibodies; single-chain antibody molecules; and multispecific antibodies formed from antibody fragments. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For e.g., such an antibody fragment may comprise on antigen binding arm linked to an Fc sequence capable of conferring in vivo stability to the fragment.

The term “hypervariable region,” “HVR,” or “HV,” when used herein refers to the regions of an antibody variable domain which are hypervariable in sequence and/or form structurally defined loops. Generally, antibodies comprise six HVRs; three in the VH(H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the hypervariable region residues as herein defined.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore eds., Springer-Verlag, New York, pp. 269-315 (1994).

An “antigen” is a predetermined antigen to which an antibody can selectively bind. The target antigen may be polypeptide, carbohydrate, nucleic acid, lipid, hapten or other naturally occurring or synthetic compound.

The term “diabodies” refers to small antibody fragments with two antigen-binding sites, which fragments comprise a heavy-chain variable domain (VH) connected to a light-chain variable domain (VL) in the same polypeptide chain (VH−VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993). Triabodies and tetrabodies are also described in Hudson et al., Nat. Med. 9:129-134 (2003).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl, Acad, Sci. USA, 103:3557-3562 (2006) regarding human antibodies generated via a human B-cell hybridoma technology.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g, Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

A “blocking” antibody or an “antagonist” antibody is one which inhibits or reduces biological activity of the antigen it binds. Certain blocking antibodies or antagonist antibodies substantially or completely inhibit the biological activity of the antigen.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

A “medicament” is an active drug to treat the disorder in question or its symptoms, or side effects.

“Tumor”, as used herein, refers to all neoplastic cell growth and proliferation, whether malignant or benign, and all pre-cancerous and cancerous cells and tissues. The terms “cancer”, “cancerous”, and “tumor” are not mutually exclusive as referred to herein.

The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulvar cancer, thyroid cancer, hepatic carcinoma, brain cancer, endometrial cancer, testis cancer, cholangiocarcinoma, gallbladder carcinoma, gastric cancer, melanoma, and various types of head and neck cancer. Dysregulation of angiogenesis can lead to many disorders that can be treated by compositions and methods of the invention. These disorders include both non-neoplastic and neoplastic conditions. Neoplastics include but are not limited those described above. Non-neoplastic disorders include but are not limited to undesired or aberrant hypertrophy, arthritis, rheumatoid arthritis (RA), psoriasis, psoriatic plaques, sarcoidosis, atherosclerosis, atherosclerotic plaques, diabetic and other proliferative retinopathies including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, pannus formation in RA, myositis ossificans, hypertropic bone formation, osteoarthritis (OA), refractory ascites, polycystic ovarian disease, endometriosis, 3rd spacing of fluid diseases (pancreatitis, compartment syndrome, burns, bowel disease), uterine fibroids, premature labor, chronic inflammation such as IBD (Crohn's disease and ulcerative colitis), renal allograft rejection, inflammatory bowel disease, nephrotic syndrome, undesired or aberrant tissue mass growth (non-cancer), hemophilic joints, hypertrophic scars, inhibition of hair growth, Osler-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, antibodies of the invention are used to delay development of a disease or disorder.

As used herein, a “week” is 7 days±2 days.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, duration of progression free survival (PFS), the response rates (RR), duration of response, and/or quality of life.

“Survival” refers to the patient remaining alive, and includes progression free survival (PFS) and overall survival (OS). Survival can be estimated by the Kaplan-Meier method, and any differences in survival are computed using the stratified log-rank test.

“Progression free survival (PFS)” refers to the time from treatment (or randomization) to first disease progression or death. For example it is the time that the patient remains alive, without return of the cancer, e.g., for a defined period of time such as about 1 month, 2 months, 3 months, 4, months, 6 months, 7 months, 8 months, 9 months, 1 year, about 2 years, about 3 years, etc., from initiation of treatment or from initial diagnosis. In one aspect Of the invention, PFS can be assessed by Response Evaluation Criteria in Solid Tumors (RECIST). In one embodiment, the PFS is extended about 6 months.

“Overall survival” refers to the patient remaining alive for a defined period of time, such as about 1 year, about 1.5 years, about 2 years, about 3 years, about 4 years, about 5 years, about 10 years, etc., from initiation of treatment or from initial diagnosis. In the studies underlying the invention the event used for survival analysis was death from any cause.

By “extending survival” or “increasing the likelihood of survival” is meant increasing PFS and/or OS in a treated patient relative to an untreated patient (i.e. relative to a patient not treated with an EGFL antagonist, e.g., an anti-EGFL7 antibody), or relative to a control treatment protocol, such as treatment only with the chemotherapeutic agent, such as those use in the standard of care for cancer. Survival is monitored for at least about one month, two months, four months, six months, nine months, or at least about 1 year, or at least about 2 years, or at least about 3 years, or at least about 4 years, or at least about 5 years, or at least about 10 years, etc., following the initiation of treatment or following the initial diagnosis.

Hazard ratio (HR) is a statistical definition for rates of events. For the purpose of the invention, hazard ratio is defined as representing the probability of an event in the experimental arm divided by the probability of an event in the control arm at any specific point in time. “Hazard ratio” in progression free survival analysis is a summary of the difference between two progression free survival curves, representing the reduction in the risk of death on treatment compared to control, over a period of follow-up.

An “anti-angiogenesis agent” or “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. For example, an anti-angiogenesis agent is an antibody or other antagonist to an angiogenic agent as defined above, e.g., antibodies to VEGF, antibodies to VEGF receptors, small molecules that block VEGF receptor signaling (e.g., PTK787/ZK2284, SU6668, SUTENT®/SU11248 (sunitinib malate), AMG706). Anti-angiogenesis agents also include native angiogenesis inhibitors, e.g., angiostatin, endostatin, etc. See, e.g., Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12):1359-1364 (1999); Tonini et al., Oncogene, 22:6549-6556 (2003) (e.g., Table 2 listing antiangiogenic factors); and, Sato Int. J. Clin. Oncol., 8:200-206 (2003) (e.g., Table 1 lists Anti-angiogenic agents used in clinical trials).

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegall (see, e.g., Nicolaou et al., Angew. Chem. Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBENEλ); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCl-779; tipifarnib (R11577); orafenib, ABT510; Bc1-2 inhibitor such as oblimersen sodium (GENASENSE®); pixantrone; EGFR inhibitors (see definition below); tyrosine kinase inhibitors (see definition below); serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releaseing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

A “growth inhibitory agent” when used herein refers to a compound or composition which inhibits growth of a cell (such as a cell expressing EGFL7) either in vitro or in vivo. Thus, the growth inhibitory agent may be one which significantly reduces the percentage of cells (such as a cell expressing EGFL7) in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Doxorubicin” is an anthracycline antibiotic. The full chemical name of doxorubicin is (8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione.

The term “Fc region-comprising polypeptide” refers to a polypeptide, such as an antibody or immunoadhesin (see definitions below), which comprises an Fc region. The C-terminal lysine (residue 447 according to the EU numbering system) of the Fc region may be removed, for example, during purification of the polypeptide or by recombinant engineering the nucleic acid encoding the polypeptide. Accordingly, a composition comprising a polypeptide having an Fc region according to this invention can comprise polypeptides with K447, with all K447 removed, or a mixture of polypeptides with and without the K447 residue.

Throughout this specification and claims, the word “comprise,” or variations such as “comprises” or “comprising,” will be understood to imply the inclusion of a stated integer or group of integers but not the exclusion of any other integer or group of integers.

### Uses

The invention provides methods and compositions useful for treating cancer, said methods comprising administration of an effective dose of an anti-EGFL7 antibody to an individual in need of such treatment. It is understood that any suitable anti-EGFL7 antibody may be used in methods of treatment, including monoclonal and/or polyclonal antibodies, a human antibody, a chimeric antibody, an affinity-matured antibody, a humanized antibody, and/or an antibody fragment.

In some embodiments, the anti-EGFL7 antibody is administered at a dose of between 1 mg/kg and 15 mg/kg. In other embodiments, the anti-EGFL7 antibody is administered at a does of about 5 mg/kg, 7.5 mg/kg or 10 mg/kg. In the methods of the invention, the anti-EGFL7 antibody may be administered at a flat dose of 374-400 mg every two weeks or 550-600 mg every three weeks. In some embodiments, the anti-EGFL7 antibody is administered at a flat dose of 400 mg every two weeks. In other embodiments, the anti-EGFL7 antibody is administered at a flat dose of 600 mg every three weeks.

In any of the methods herein, one may administer to the subject or patient along with the antibody herein an effective amount of a second medicament (where the antibody herein is a first medicament), which is another active agent that can treat the condition in the subject that requires treatment. For instance, an antibody of the invention may be co-administered with another antibody, chemotherapeutic agent(s) (including cocktails of chemotherapeutic agents), anti-angiogenic agent(s), immunosuppressive agents(s), cytokine(s), cytokine antagonist(s), and/or growth-inhibitory agent(s). The type of such second medicament depends on various factors, including the type of disorder, the severity of the disease, the condition and age of the patient, the type and dose of first medicament employed, etc.

Where an antibody of the invention inhibits tumor growth, for example, it may be particularly desirable to combine it with one or more other therapeutic agents that also inhibit tumor growth. For instance, an antibody of the invention may be combined with an anti-angiogenic agent, such as an anti-VEGF antibody (e.g., AVASTIN®) and/or anti-ErbB antibodies (e.g. HERCEPTIN® trastuzumab anti-HER2 antibody or an anti-HER2 antibody that binds to Domain II of HER2, such as OMNITARG™ pertuzumab anti-HER2 antibody) in a treatment scheme, e.g. in treating any of the disease described herein, including colorectal cancer, lung cancer, hepatocellular carcinoma, breast cancer and/or pancreatic cancer. In some instances, the previous combinations may be accomplished using a bispecific antibody. Alternatively, or additionally, the patient may receive combined radiation therapy (e.g. external beam irradiation or therapy with a radioactive labeled agent, such as an antibody). Such combined therapies noted above include combined administration (where the two or more agents are included in the same or separate formulations), and separate administration, in which case, administration of the antibody of the invention can occur prior to, and/or following, administration of the adjunct therapy or therapies. In addition, combining an antibody of this invention with a relatively non-cytotoxic agent such as another biologic molecule, e.g., another antibody is expected to reduce cytotoxicity versus combining the antibody with a chemotherapeutic agent of other agent that is highly toxic to cells.

Treatment with a combination of the antibody herein with one or more second medicaments preferably results in an improvement in the signs or symptoms of cancer. For instance, such therapy may result in an improvement in survival (overall survival and/or progression-free survival) relative to a patient treated with the second medicament only (e.g., a chemotherapeutic agent only), and/or may result in an objective response *(partial or complete, preferably complete). Moreover, treatment with the combination of an antibody herein and one or more second medicament(s) preferably results in an additive, and more preferably synergistic (or greater than additive), therapeutic benefit to the patient. Preferably, in this combination method the timing between at least one administration of the second medicament and at least one administration of the antibody herein is about one month or less, e.g about two weeks or less.

For treatment of cancers, the second medicament is preferably another antibody, chemotherapeutic agent (including cocktails of chemotherapeutic agents), anti-angiogenic agent, immunosuppressive agent, prodrug, cytokine, cytokine antagonist, cytotoxic radiotherapy, corticosteroid, anti-emetic, cancer vaccine, analgesic, anti-vascular agent, and/or growth-inhibitory agent. The cytotoxic agent includes an agent interacting with DNA, the antimetabolites, the topoisomerase I or II inhibitors, or the spindle inhibitor or stabilizer agents (e.g., preferably vinca alkaloid, more preferably selected from vinblastine, deoxyvinblastine, vincristine, vindesine, vinorelbine, vinepidine, vinfosiltine, vinzolidine and vinfunine), or any agent used in chemotherapy such as 5-FU, a taxane, doxorubicin, or dexamethasone.

In some embodiments, the second medicament is another antibody used to treat cancers such as those directed against the extracellular domain of the HER2/neu receptor, e.g., trastuzumab, or one of its functional fragments, pan-HER inhibitor, a Src inhibitor, a MEK inhibitor, or an EGFR inhibitor (e.g., an anti-EGFR antibody (such as one inhibiting the tyrosine kinase activity of the EGFR), which is preferably the mouse monoclonal antibody 225, its mouse-man chimeric derivative C225, or a humanized antibody derived from this antibody 225 or derived natural agents, dianilinophthalimides, pyrazolo- or pyrrolopyridopyrimidines, quinazilines, gefitinib, erlotinib, cetuximab, ABX-EFG, canertinib, EKB-569 and PKI-166), or dual-EGFR/HER-2 inhibitor such as lapatanib. Additional second medicaments include alemtuzumab (CAMPATHT™), FavID (IDKLH), CD20 antibodies with altered glycosylation, such as GA-101/GLYCARTT™, oblimersen (GENASENSET™), thalidomide and analogs thereof, such as lenalidomide (REVLIMIDT™), imatinib, sorafenib, ofatumumab (HUMAX-CD20™), anti-CD40 antibody, e.g. SGN-40, and anti-CD-80 antibody, e.g. galiximab.

The anti-emetic agent is preferably ondansetron hydrochloride, granisetron hydrochloride, metroclopramide, domperidone, haloperidol, cyclizine, lorazepam, prochlorperazine, dexamethasone, levomepromazine, or tropisetron. The vaccine is preferably GM-CSF DNA and cell-based vaccines, dendritic cell vaccine, recombinant viral vaccines, heat shock protein (HSP) vaccines, allogeneic or autologous tumor vaccines. The analgesic agent preferably is ibuprofen, naproxen, choline magnesium trisalicylate, or oxycodone hydrochloride. The anti-vascular agent preferably is bevacizumab, or rhuMAb-VEGF. Further second medicaments include anti-proliferative agents such a farnesyl protein transferase inhibitors, anti-VEGF inhibitors, p53 inhibitors, or PDGFR inhibitors. The second medicament herein includes also biologic-targeted therapy such as treatment with antibodies as well as small-molecule-targeted therapy, for example, against certain receptors.

Many anti-angiogenic agents have been identified and are known in the art, including those listed herein, e.g., listed under Definitions, and by, e.g., Carmeliet and Jain, Nature 407:249-257 (2000); Ferrara et al., Nature Reviews:Drug Discovery, 3:391-400 (2004); and Sato Int. J. Clin. Oncol., 8:200-206 (2003). See also, US Patent Application US20030055006. In one embodiment, an anti-EGFL7 antibody is used in combination with an anti-VEGF neutralizing antibody (or fragment) and/or another VEGF antagonist or a VEGF receptor antagonist including, but not limited to, for example, soluble VEGF receptor (e.g., VEGFR-1, VEGFR-2, VEGFR-3, neuropilins (e.g., NRP1, NRP2)) fragments, aptamers capable of blocking VEGF or VEGFR, neutralizing anti-VEGFR antibodies, low molecule weight inhibitors of VEGFR tyrosine kinases (RTK), antisense strategies for VEGF, ribozymes against VEGF or VEGF receptors, antagonist variants of VEGF; and any combinations thereof. Alternatively, or additionally, two or more angiogenesis inhibitors may optionally be co-administered to the patient in addition to VEGF antagonist and other agent. In certain embodiment, one or more additional therapeutic agents, e.g., anti-cancer agents, can be administered in combination with anti-EGFL7 antibody, the VEGF antagonist, and an anti-angiogenesis agent.

Chemotherapeutic agents useful herein are described supra, e.g., in the definition of “chemotherapeutic agent”.

Such second medicaments may be administered within 48 hours after the antibodies herein are administered, or within 24 hours, or within 12 hours, or within 3-12 hours after said agent, or may be administered over a pre-selected period of time, which is preferably about 1 to 2 days. Further, the dose of such agent may be sub-therapeutic.

The antibodies herein can be administered concurrently, sequentially, or alternating with the second medicament or upon non-responsiveness with other therapy. Thus, the combined administration of a second medicament includes co-administration (concurrent administration), using separate formulations or a single pharmaceutical formulation, and consecutive administration in either order, wherein preferably there is a time period while both (or all) medicaments simultaneously exert their biological activities. All these second medicaments may be used in combination with each other or by themselves with the first medicament, so that the express “second medicament” as used herein does not mean it is the only medicament besides the first medicament, respectively. Thus, the second medicament need not be one medicament, but may constitute or comprise more than one such drug.

These second medicaments as set forth herein are generally used in the same dosages and with administration routes as the first medicaments, or about from 1 to 99% of the dosages of the first medicaments. If such second medicaments are used at all, preferably, they are used in lower amounts than if the first medicament were not present, especially in subsequent dosings beyond the initial dosing with the first medicament, so as to eliminate or reduce side effects caused thereby.

The invention provides a method for treating lung cancer, e.g., non-small cell lung cancer (NSCLC) in a patient comprising a dosing regimen comprising treatment cycles, wherein the patient is administered, on day 1 of each cycle, 200 mg/m2 paclitaxel, carboplatin (AUC of 6 mg/ml), 15 mg/kg bevacizumab and 600 mg of an anti-EGFL7 antibody, wherein each cycle is repeated every 21 days. In some embodiments, the paclitaxel and carboplatin are administered until disease progression or for up to 6 cycles. In some embodiments, the anti-EGFL7 and/or the bevacizumab are administered to the patient until disease progression or for up to 34 cycles.

The invention also provides a method for treating lung cancer, e.g., NSCLC in a patient comprising a dosing regimen comprising treatment cycles, wherein the patient is administered on day 1 of each of four cycles carboplatin (AUC of 6 mg/ml), pemetrexed 500 mg/m2, and 15 mg/kg bevacizumab and 600 mg anti-EGFL7 antibody, wherein the patient is administered on day 1 of each subsequent cycle, pemetrexed 500 mg/m2, and 15 mg/kg bevacizumab and 600 mg anti-EGFL7 antibody, wherein each cycle is repeated every 21 days. In some embodiments the anti-EGFL7 and/or bevacizumab are administered to the patient until disease progression.

The invention also provides a method for treating colorectal cancer in a patient comprising a dosing regimen comprising treatment cycles, wherein the patient is administered, on day 1 of the first cycle, 85 mg/m2 oxaliplatin, 400 mg/m25-fluorouracil (5-FU), 400 mg/m2 folinic acid, 5 mg/kg bevacizumab, and 400 mg anti-EGFL7 antibody, wherein the patient is administered on day 1 of each subsequent cycle, 85 mg/m2 oxaliplatin, 2400 mg/m2 5-FU, 400 mg/m2 folinic acid, 5 mg/kg bevacizumab, and 400 mg of an anti-EGFL7 antibody, wherein each cycle is repeated every 14 days. In some embodiments, the oxaliplatin is administered for up to 8 cycles. In some embodiments the 5-FU, folinic acid, bevacizumab and/or anti-EGFL7 antibody is administered until disease progression or for up to 52 cycles.

The antibodies of the invention (and adjunct therapeutic agent) is/are administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the antibodies are suitably administered by pulse infusion, particularly with declining doses of the antibody. Dosing can be by any suitable route, e.g. by injections, such as intravenous or subcutaneous injections, depending in part on whether the administration is brief or chronic.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, etc. The containers may be formed from a variety of materials such as glass or plastic. The container holds a composition which is by itself or when combined with another composition(s) effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice, such as cancer. Moreover, the article of manufacture may comprise (a) a first container with a composition contained therein, wherein the composition comprises an antibody of the invention; and (b) a second container with a composition contained therein. The article of manufacture in this embodiment of the invention may further comprise a package insert indicating that the first and second antibody compositions can be used to treat a particular condition, e.g. cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The following are examples of the methods and compositions of the invention. It is understood that various other embodiments may be practiced, given the general description provided above.

### Example 1

Anti-EGFL7 Enhancement of Anti-VEGF Activity is Dose-Dependent

This example demonstrates that anti-EGFL7 therapy enhances the overall survival benefit of anti-VEGF therapy, that the enhancement is dose-dependent, and that the optimal dose is 1 mg/kg twice per week in a mouse, which corresponds to about 5 mg/kg every two weeks in human. We tested the ability of anti-EGFL7 MAb to enhance anti-VEGF activity using a “K-ras^{G12D};;p53-null”-driven NSCLC GEMM (Johnson et al (2001) Nature 410:1111-16; Jackson et al (2001) Genes & Development 15:3243-48; Singh et al (2010) Nature Biotechnology 28:585-93). Tumor bearing mice were subjected to computerized tomography scan to evaluate tumor burdens, and were randomized into five groups with equal mean tumor burdens. We administered anti-VEGF MAb (mB20-4.1.1; WO 2009/073160, filed Dec. 1, 2008) alone or in combination with the murine anti-EGFL7 antibody 18F7 (mul8F7; US-2010-0285009-A1, filed May 7, 2010) into tumor bearing mice. The anti-VEGF antibody (or control anti-ragweed antibody) was administered twice per week at 5 mg/kg and the anti-EGFL7 antibody was administered twice per week at 0.1 mg/kg, 1.0 mg/kg, and 10 mg/kg. As shown in Table 1, administration of anti-EGFL7 antibody enhanced the activity of anti-VEGF therapy at all doses tested, but, interestingly, the optimal dose occurs at an intermediate anti-EGFL7 dose of 1 mg/kg.

TABLE 1
Anti-EGFL7 Enhancement of Anti-VEGF Activity

| Antibody | No. of Mice | p-value | HR |
|---|---|---|---|
| B20-4.1.1 (5 mg/kg) | 53 | — | — |
| B20-4.1.1 (5 mg/kg) + | 20 | 0.9722 | 0.79 |
| m18F17 (0.1 mg/kg) | | | |
| B20-4.1.1 (5 mg/kg) + | 46 | 0.0130 | 0.56 |
| m18F17 (1 mg/kg) | | | |
| B20-4.1.1 (5 mg/kg) + | 37 | 0.4600 | 0.55 |
| m18F17 (10 mg/kg) | | | |

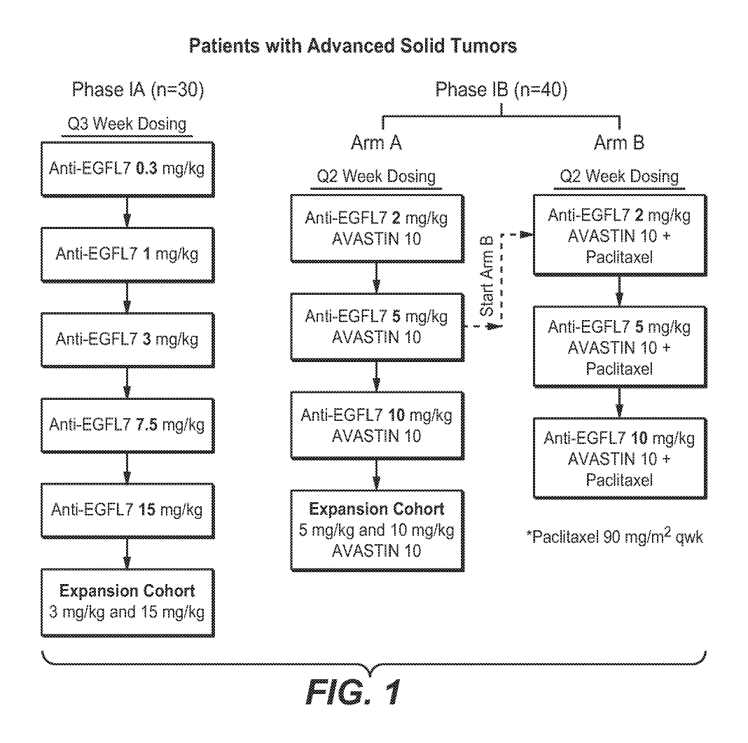

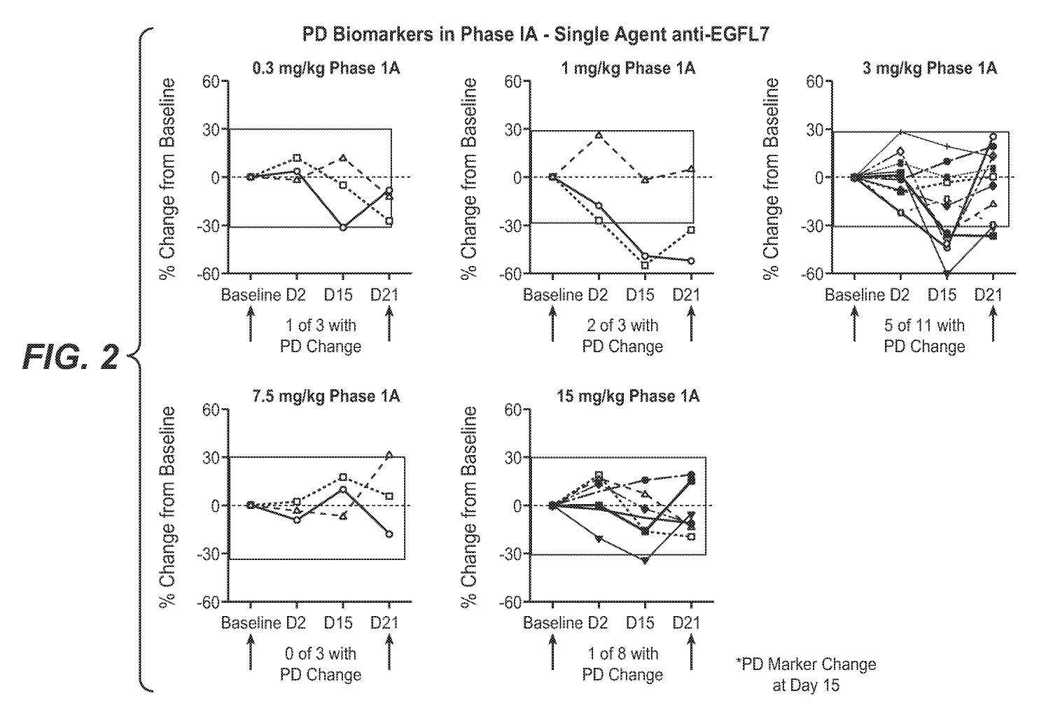

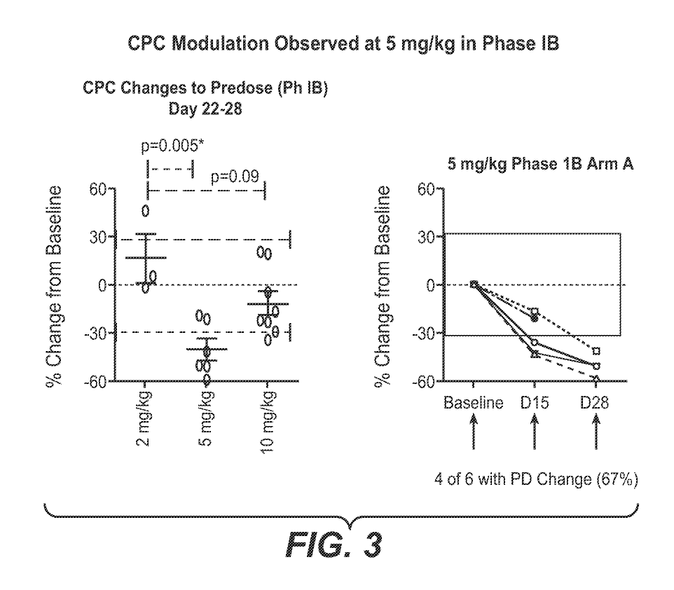

We observed that a population of circulating progenitor cells (CD34^{Hi}CD31^{Low}CD45^{Low}; “CPCs”) in mouse tumor models is reduced following anti-EGFL7 treatment. These cells are readily isolated and quantified from blood and we measured CPCs as a pharmacodynamic (PD) biomarker in samples from human patients enrolled in Phase Ia and Phase Ib, dose-escalation and expansion clinical trials. The design of these Phase Ia and Phase Ib trials is outlined in FIG. 1. Blood from patients was collected in EDTA tubes. CPCs were measured with a lyse/no wash flow cytometry method in Trucount™ tubes using 100 ul of whole blood. The following antibody conjugates were used for staining: CD45-APC-Cy7, CD34-APC, CD31-PE-Cy7 and CD133-PE. A nuclear dye (Syto® 16) was included to distinguish cells from debris and appropriate isotype controls were used to set gates. Samples were acquired on FACSCanto™ II cytometer and analyzed using FACSDiva™ software (BD Biosciences, CA). Consistent with the efficacy data observed in Example 1, we observed in the human patients that the maximal change in CPC levels occurred at the 5 mg/kg dose level (FIGS. 2 and 3).

We also incorporate dynamic contrast-enhanced magnetic resonance imaging (DCE-MRI) as a PD marker in the Phase Ia and Phase Ib clinical trails outlined in FIG. 1. Two baseline scans were obtained prior to dosing on Cycle 1 Day 1 (C1D1) to assess reproducibility and on-treatment DCE-MR1s were performed on C1D3 and C1D13 in Phase Ib (dosing was every two weeks). Data were fit to a kinetic model to derive median values of vascular parameters such as Ktrans and a linear mixed effects model was used to summarize results. Maximal changes in DCE-MRI measurements occurred at the 5 mg/kg dose, consistent with the other results.

Taken together, the PD data support dosing of anti-EGFL7 at 5 mg/kg every two weeks in humans. In addition, we observed that the antibody exhibited linear pharmacokinetic properties typical of IgG1 monoclonal antibodies, with an elimination half-life of approximately 2 weeks. These PK data support dosing every 2 or 3 weeks to maintain desired drug exposure and fixed dosing equivalent to the optimal dosing of 5 mg/kg every two weeks (e.g. 375-400 mg every two weeks or 550-600 mg every three weeks).

### Example 2

Study Evaluating the Safety and Efficacy of Anti-EGFL7 Antibody in Combination with Carboplatin, Paclitaxel and Bevacizumab in Patients with Advanced or Recurrent Non-Squamous Non-Small Cell Lung Cancer Who have not Received Prior Chemotherapy for Advanced Disease

A Phase II, multicenter, randomized, double-blind, placebo-controlled trial will be conducted to evaluate the efficacy and safety of anti-EGFL7 antibody combined with paclitaxel+carboplatin+bevacizumab (anti-VEGF) therapy in patients with histologically or cytologically documented inoperable, locally advanced, metastatic (Stage IV), or recurrent non-squamous NSCLC. Approximately 100 patients who meet the study selection criteria (see below) will be enrolled and randomized to one of two treatment arms:
Control arm: paclitaxel+carboplatin+bevacizumab plus placebo
Experimental arm: paclitaxel+carboplatin+bevacizumab plus anti-EGFL7 antibody Study treatment will be given in cycles repeated every 21 days and will consist of paclitaxel (200 mg/m2) administered IV on Day 1, carboplatin (AUC of 6 mg/mL min by the Calvert formula) administered IV on Day 1, and bevacizumab (15 mg/kg) administered IV on Day 1. Patients in the experimental arm will receive anti-EGFL7 antibody at a fixed dose of 600 mg IV on Day 1 of Cycle 1, followed by subsequent doses of 600 mg every 21 days. Patients in the control arm will receive an equivalent volume of placebo according to the same schedule. The preferred order of study treatment administration will be paclitaxel, carboplatin, and bevacizumab, followed by anti-EGFL7/placebo. Paclitaxel and carboplatin will be administered until disease progression or unacceptable toxicity, for a maximum of 6 cycles; bevacizumab and anti-EGFL7/placebo will be administered until disease progression or unacceptable toxicity, for a maximum of 24 months (34 cycles). Upon treatment discontinuation, patients will be followed approximately every 3 months for survival.

Primary Outcome Measure is Progression-free survival (defined as the time from randomization to the first occurrence of progression based on RECIST 1.1 criteria or death from any cause on study). Secondary Outcome Measures include Objective response (partial response plus complete response) as determined by the Investigator using RECIST 1.1, Duration of objective response (defined as the first occurrence of a documented objective response until the time of progression or death from any cause on study), and Overall survival (defined as the time from randomization until death from any cause).

For selection of patients, inclusion criteria include histologically or cytologically documented inoperable (Stage 1V) or recurrent non-squamous NSCLC. Diagnoses of non-squamous NSCLC that are based on sputum cytology alone are not acceptable (mixed tumors should be categorized according to the predominant cell type), ECOG performance status of 0 or 1, life expectancy>12 weeks, measurable disease, as defined by RECIST 1.1, and adequate hematologic and end organ function. Exclusion criteria include prior therapy (including chemotherapy, antibody therapy, tyrosine kinase inhibitors, radiotherapy, immunotherapy, hormonal therapy or investigational therapy) before Day 1 of Cycle 1 for the treatment of Stage 1V or recurrent NSCLC (patients who received prior adjuvant chemotherapy or radiotherapy for NSCLC are not excluded if the time interval from completion of adjuvant therapy until disease progression is >12 months), treatment with any other investigational agent or participation in another clinical trial with therapeutic intent within 28 days prior to enrollment, malignancies other than NSCLC within 5 years prior to randomization, except for adequately treated carcinoma in situ of the cervix, basal or squamous cell skin cancer, localized prostate cancer treated surgically with curative intent, ductal carcinoma in situ treated surgically with curative intent, pregnant and lactating women, and active infection requiring IV antibiotics. Bevacizumab-specific exclusions include histologically or cytologically documented inoperable, locally advanced, mixed non-small cell and small cell tumors or mixed adenosquamous carcinomas with a predominant squamous component, evidence of tumor invading major blood vessels on imaging, evidence of CNS metastases, history of stroke or TIAs within 6 months prior to Day 1 of treatment, significant vascular disease within 6 months prior to Day 1, and major surgical procedure, open biopsy, or significant traumatic injury within 28 days prior to Day 1.

### Example 3

Study Evaluating the Safety and Efficacy of Anti-EGFL7 Antibody in Combination with FOLFOX and Bevacizumab in Patients With Previously Untreated Metastatic Colorectal Cancer

A Phase II, multicenter, randomized, double-blind, placebo-controlled trial will be conducted to evaluate the efficacy and safety of anti-EGFL7 antibody combined with modified FOLFOX-6 (mFOLFOX-6)+bevacizumab (anti-VEGF) therapy in patients with metastatic colorectal cancer (mCRC). Approximately 120 patients who meet the study selection criteria (see below) will be enrolled and randomized to one of two treatment arms:

Control arm: mFOLFOX-6+ bevacizumab plus placebo
Experimental arm: mFOLFOX-6+ bevacizumab plus MEGF0444A

Study treatment will be given in cycles repeated every 14 days and will consist of mFOLFOX-6+ beyacizumab. Patients in the experimental arm will additionally receive anti-EGFL7 antibody at a fixed dose of 400 mg IV on Day 1 of Cycle 1, followed by subsequent doses of 400 mg every 14 days. Patients in the control arm will receive an equivalent volume of placebo according to the same schedule. Oxaliplatin at a starting dose of 85 mg/m2 will be administered up to 8 cycles. 5-fluorouracil (5-FU; starting bolus and infusional doses of 400 mg/m2 and 2400 mg/m2, respectively), folinic acid (starting dose of 400 mg/m2), bevacizumab (5 mg/kg) and anti-EGFL7 antibody/placebo will be administered until disease progression or unacceptable toxicity, for a maximum of 24 months (up to 52 cycles). If patients stop chemotherapy either in part or in whole, then they will continue on bevacizumab and anti-EGFL7 antibody/placebo until documented disease progression or unacceptable toxicity, for a maximum of 24 months.

Primary Outcome Measure is Progression-free survival (defined as the time from randomization to the first occurrence of progression based on RECIST 1.1 criteria or death from any cause on study). Secondary Outcome Measures include Objective response (partial response plus complete response) as determined by the Investigator using RECIST 1.1, Duration of objective response (defined as the first occurrence of a documented objective response until the time of progression or death from any cause on study), and Overall survival (defined as the time from randomization until death from any cause).

For selection of patients, inclusion criteria include histologically or cytologically confirmed colorectal cancer (CRC) not amenable to potentially curative resection with at least one measurable metastatic lesion, as defined by RECIST v1.1., ECOG performance status of 0 or 1, and adequate hematologic and end organ function. Exclusion criteria include prior systemic therapy (including chemotherapy, antibody therapy, tyrosine kinase inhibitors, radiotherapy, immunotherapy, hormonal therapy or investigational therapy) before Day 1 of Cycle 1 for the treatment of mCRC, treatment with any other investigational agent or participation in another clinical trial with therapeutic intent within 28 days prior to Day 1 of Cycle 1, malignancies other than CRC within 5 years prior to randomization, except for those with a negligible risk of metastasis or death, Lactating women, clinically suspected or confirmed CNS metastases or carcinomatous meningitis, active infection requiring IV antibiotics, active autoimmune disease that is not controlled by nonsteroidal anti-inflammatory drugs, inhaled corticosteroids, or the equivalent of â [symbol] 10 mg/day prednisone, known clinically significant liver disease, including active viral, alcoholic, or other hepatitis, or cirrhosis. Bevacizumab-specific exclusions include history of stroke or TIAs within 6 months prior to Day 1 of treatment, significant vascular disease within 6 months prior to Day 1, and major surgical procedure, open biopsy, or significant traumatic injury within 28 days prior to Day 1.

1. A method for the treatment of cancer in a human patient comprising administering an anti-EGFL7 antibody, the method comprising administering the antibody at a dose of between 1 mg/kg and 15 mg/kg.

2. A method for the treatment of cancer in a human patient comprising administering an anti-EGFL7 antibody, the method comprising administering the antibody at a flat dose selected from the group consisting of: (a) 375-400 mg every two weeks and (b) 550-600 mg every three weeks.

3-4. (canceled)

5. A method for the treatment of cancer in a human patient comprising administering an anti-EGFL7 antibody, the method comprising administering a first dose of the anti-EGFL7 antibody to the patient and a second dose of the anti-EGFL7 antibody to the patient, wherein the first dose and the second dose are each between 1 mg/kg and 15 mg/kg and the second dose follows the first does by between 1 and 4 weeks.

6-8. (canceled)

9. The method of any one of claims 1, 2 or 5, wherein said anti-EGFL7 antibody comprises a variable domain comprising the following HVR sequences:
(i)
HVR-L1 comprising
(SEQ ID NO: 1)
KASQSVDYSGDSYMS;
(ii)
HVR-L2 comprising
(SEQ ID NO: 2)
GASYRES;
(iii)
HVR-L3 comprising
(SEQ ID NO: 3)
QQNNEEPYT;
(iv)
HVR-H1 comprising
(SEQ ID NO: 4)
GHTFTTYGMS;
(v)
HVR-H2 comprising
(SEQ ID NO: 5)
GWINTHSGVPTYADDFKG;
and
(vi)
HVR-H3 comprising
(SEQ ID NO: 6)
LGSYAVDY.

10. The method of claim 9, wherein said anti-EGFL7 antibody comprises a heavy chain variable region sequence selected from the group consisting of:
(SEQ ID NO: 7)
EVQLVESGGGLVQPGGSLRLSCAASGHTFTTYGMSWVRQAPGKGLEWV
GWINTHSGVPTYADDFKGRFTISLDNSKNTAYLQMNSLRAEDTAVYYC
ARLGSYAVDYWGQGTLVTVSS;
and
(SEQ ID NO: 8)
EIQLVESGGGLVQPGGSLRLSCAASGHTFTTYGMSWVRQAPGKGLEWM
GWINTHSGVPTYADDFKGRFTISLDNSKSTAYLQMNSLRAEDTAVYFC
ARLGSYAVDYWGQGTLVTVSS.

11. (canceled)

12. The method of claim 9 wherein said anti-EGFL7 antibody comprises the following light chain variable region sequence:
(SEQ ID NO: 9)
DIQMTQSPSSLSASVGDRVTITCKASQSVDYSGDSYMSWYQQKPGKAP
KLLIYGASYRESGVPSRFSGSGSGTDFTLTISSLQPEDFATYYCQQNN
EEPYTFGQGTKVEIKR.

13. The method of any one of claims 1, 2 or 5, wherein said anti-EGFL7 antibody comprises a variable domain comprising the following HVR sequences:
(i)
HVR-L1 comprising
(SEQ ID NO: 10)
RTSQSLVHINGITYLH;
(ii)
HVR-L2 comprising
(SEQ ID NO: 11)
RVSNRFS;
(iii)
HVR-L3 comprising
(SEQ ID NO: 12)
GQSTHVPLT;
(iv)
HVR-H1 comprising
(SEQ ID NO: 13)
GYTFIDYYMN;
(v)
HVR-H2 comprising
(SEQ ID NO: 14)
GDINLDNGGTHYNQKFKG;
and
(vi)
HVR-H3 comprising
(SEQ ID NO: 15)
AREGVYHDYDDYAMDY.

14. The method of claim 13, wherein said anti-EGFL7 antibody comprises the following heavy chain variable region sequence:
(SEQ ID NO: 16)
EVQLVESGGGLVQPGGSLRLSCAASGYTFIDYYMNWVRQAPGKGLEWVG
DINLDNGGTHYNQKFKGRFTISRDKSKNTAYLQMNSLRAEDTAVYYCAR
EGVYHDYDDYAMDYWGQGTLVTVSS.

15. The method of claim 13, wherein said anti-EGFL7 antibody comprises the following light chain variable region sequence:
(SEQ ID NO: 17)
DIQMTQSPSSLSASVGDRVTITCRTSQSLVHINGITYLHWYQQKPGKAP
KLLIYRVSNRFSGVPSRFSGSGSGTDFTLTISSLQPEDFATYYCGQSTH
VPLTFGQGTKVEIKR.

16. The method of any one of claim 1, 2 or 5, wherein said anti-EGFL7 antibody comprises a variable domain comprising the following HVR sequences:
(i)
HVR-L1 comprising
(SEQ ID NO: 18)
RTSQSLVHINAITYLH;
(ii)
HVR-L2 comprising
(SEQ ID NO: 11)
RVSNRFS;
(iii)
HVR-L3 comprising
(SEQ ID NO: 12)
GQSTHVPLT;
(iv)
HVR-H1 comprising
(SEQ ID NO: 13)
GYTFIDYYMN;
(v)
HVR-H2 comprising
(SEQ ID NO: 19)
GDINLDNSGTHYNQKFKG;
and
(vi)
HVR-H3 comprising
(SEQ ID NO: 15)
AREGVYHDYDDYAMDY.

17. The method of claim 16, wherein said anti-EGFL7 antibody comprises the following heavy chain variable region sequence:
(SEQ ID NO: 20)
EVQLVESGGGLVQPGGSLRLSCAASGYTFIDYYMNWVRQAPGKGLEWVG
DINLDNSGTHYNQKFKGRFTISRDKSKNTAYLQMNSLRAEDTAVYYCAR
EGVYHDYDDYAMDYWGQGTLVTVSS.

18. The method of claim 16, wherein said anti-EGFL7 antibody comprises the following light chain variable region sequence:
(SEQ ID NO: 21)
DIQMTQSPSSLSASVGDRVTITCRTSQSLVHINAITYLHWYQQKPGKAP
KLLIYRVSNRFSGVPSRFSGSGSGTDFTLTISSLQPEDFATYYCGQSTH
VPLTFGQGTKVEIKR.

19. The method of any one of claims 1, 2 or 5, wherein the antibody is administered in an infusion of 10, 20 or 30 minutes.

20. The method of any one of claims 1, 2 or 5, further comprising the step of administering another anti-angiogenic agent.

21. The method of claim 20, wherein the other anti-angiogenic agent is an anti-vascular endothelial growth factor (VEGF) antagonist.

22. The method of claim 21, wherein said anti-VEGF antagonist is an anti-VEGF antibody.

23. The method of claim 22, wherein said anti-VEGF antibody is bevacizumab.

24. The method of any one of claims 1, 2 or 5, wherein said anti-EGFL7 antibody is a bispecific antibody.

25. The method of claim 24, wherein said bispecific antibody binds to VEGF.

26. The method of claim 24, where said bispecific antibody binds to the same VEGF epitope as bevacizumab.

27. The method of any one of claim 1, 2 or 5, wherein said cancer is selected from group consisting of breast cancer, leukemia, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colon cancer, colorectal cancer, endometrial carcinoma, salivary gland carcinoma, kidney cancer, liver cancer, prostate cancer, vulvar cancer, thyroid cancer, hepatic carcinoma and various types of head and neck cancer.

28. The method of claim 27, wherein said cancer is breast cancer, NSCLC or CRC.

29. The method of any one of claim 1, 2 or 5, further comprising administering an effective amount of a chemotherapeutic agent.

30. An article of manufacture comprising a container, a composition within the container comprising an anti-EGFL7 antibody, and a label or package insert with instructions to administer the antibody at a dose of between 1 mg/kg and 15 mg/kg.

31. An article of manufacture comprising a container, a composition within the container comprising an anti-EGFL7 antibody, and a label or package insert with instructions to administer a first dose of an anti-EGFL7 antibody to the patient and a second dose of an anti-EGFL7 antibody to the patient, wherein the first dose and the second dose are each between 1 mg/kg and 15 mg/kg and the second dose follows the first does by between 1 and 4 weeks.

32-34. (canceled)

35. An article of manufacture comprising a container, a composition within the container comprising an anti-EGFL7 antibody, and a label or package insert with instructions to administer the antibody at a flat dose selected from the group consisting of: (a) 375-400 mg every two weeks and (b) 550-600 mg every three weeks.

36-37. (canceled)

38. A method for the treatment of NSCLC in a human patient, comprising a dosing regimen comprising treatment cycles, wherein the patient is administered, on day 1 of each cycle, 200 mg/m2 pactlitaxel, carboplatin (AUC of 6 mg/ml min), 15 mg/kg bevacizumab, and 600 mg of an anti-EGFL7 antibody, each cycle being repeated every 21 days.

39-40. (canceled)

41. A method for the treatment of colorectal cancer in a human patient, comprising a dosing regimen comprising treatment cycles, wherein the patient is administered, on day 1 of the first cycle, 85 mg/m2 oxaliplatin, 400 mg/m25-fluorourcail (5-FU), 400 mg/m2 folinic acid, 5 mg/kg bevacizumab, and 400 mg of an anti-EGFL7 antibody, and wherein the patient is administered on day 1 of each subsequent cycle, 85 mg/m2 oxaliplatin, 2400 mg/m2 5-FU, 400 mg/m2 folinic acid, 5 mg/kg bevacizumab, and 400 mg of an anti-EGFL7 antibody, each cycle being repeated every 14 days.

42-43. (canceled)

44. The method of any one of claims 38 and 41, wherein said anti-EGFL7 antibody comprises a variable domain comprising the following HVR sequences:
(i)
HVR-L1 comprising
(SEQ ID NO: 1)
KASQSVDYSGDSYMS;
(ii)
HVR-L2 comprising
(SEQ ID NO: 2)
GASYRES;
(iii)
HVR-L3 comprising
(SEQ ID NO: 3)
QQNNEEPYT;
(iv)
HVR-H1 comprising
(SEQ ID NO: 4)
GHTFTTYGMS;
(v)
HVR-H2 comprising
(SEQ ID NO: 5)
GWINTHSGVPTYADDFKG;
and
(vi)
HVR-H3 comprising
(SEQ ID NO: 6)
LGSYAVDY.

45. The method of claim 44, wherein said anti-EGFL7 antibody comprises a heavy chain variable region sequence selected from the group consisting of:
(SEQ ID NO: 7)
EVQLVESGGGLVQPGGSLRLSCAASGHTFTTYGMSWVRQAPGKGLEWV
GWINTHSGVPTYADDFKGRFTISLDNSKNTAYLQMNSLRAEDTAVYYC
ARLGSYAVDYWGQGTLVTVSS;
and
(SEQ ID NO: 8)
EIQLVESGGGLVQPGGSLRLSCAASGHTFTTYGMSWVRQAPGKGLEWM
GWINTHSGVPTYADDFKGRFTISLDNSKSTAYLQMNSLRAEDTAVYFC
ARLGSYAVDYWGQGTLVTVSS.

46. (canceled)

47. The method of claim 44, wherein said anti-EGFL7 antibody comprises the following light chain variable region sequence:
(SEQ ID NO: 9)
DIQMTQSPSSLSASVGDRVTITCKASQSVDYSGDSYMSWYQQKPGKAP
KLLIYGASYRESGVPSRFSGSGSGTDFTLTISSLQPEDFATYYCQQNN
EEPYTFGQGTKVEIKR.

48. The method of any one of claims 38 and 41, wherein said anti-EGFL7 antibody comprises a variable domain comprising the following HVR sequences:
(i)
HVR-L1 comprising
(SEQ ID NO: 10)
RTSQSLVHINGITYLH;
(ii)
HVR-L2 comprising
(SEQ ID NO: 11)
RVSNRFS;
(iii)
HVR-L3 comprising
(SEQ ID NO: 12)
GQSTHVPLT;
(iv)
HVR-H1 comprising
(SEQ ID NO: 13)
GYTFIDYYMN;
(v)
HVR-H2 comprising
(SEQ ID NO: 14)
GDINLDNGGTHYNQKFKG;
and
(vi)
HVR-H3 comprising
(SEQ ID NO: 15)
AREGVYHDYDDYAMDY.

49. The method of claim 48, wherein said anti-EGFL7 antibody comprises the following heavy chain variable region sequence:
(SEQ ID NO: 16)
EVQLVESGGGLVQPGGSLRLSCAASGYTFIDYYMNWVRQAPGKGLEWV
GDINLDNGGTHYNQKFKGRFTISRDKSKNTAYLQMNSLRAEDTAVYYC
AREGVYHDYDDYAMDYWGQGTLVTVSS.

50. The method of claim 48, wherein said anti-EGFL7 antibody comprises the following light chain variable region sequence:
(SEQ ID NO: 17)
DIQMTQSPSSLSASVGDRVTITCRTSQSLVHINGITYLHWYQQKPGKA
PKLLIYRVSNRFSGVPSRFSGSGSGTDFTLTISSLQPEDFATYYCGQS
THVPLTFGQGTKVEIKR.

51. The method of any one of claims 38 and 41, wherein said anti-EGFL7 antibody comprises a variable domain comprising the following HVR sequences:
(i)
HVR-L1 comprising
(SEQ ID NO: 18)
RTSQSLVHINAITYLH;
(ii)
HVR-L2 comprising
(SEQ ID NO: 11)
RVSNRFS;
(iii)
HVR-L3 comprising
(SEQ ID NO: 12)
GQSTHVPLT;
(iv)
HVR-H1 comprising
(SEQ ID NO: 13)
GYTFIDYYMN;
(v)
HVR-H2 comprising
(SEQ ID NO: 19)
GDINLDNSGTHYNQKFKG;
and
(vi)
HVR-H3 comprising
(SEQ ID NO: 15)
AREGVYHDYDDYAMDY.

52. The method of claim 51, wherein said anti-EGFL7 antibody comprises the following heavy chain variable region sequence:
(SEQ ID NO: 20)
EVQLVESGGGLVQPGGSLRLSCAASGYTFIDYYMNWVRQAPGKGLEW
VGDINLDNSGTHYNQKFKGRFTISRDKSKNTAYLQMNSLRAEDTAVY
YCAREGVYHDYDDYAMDYWGQGTLVTVSS.

53. The method of claim 51, wherein said anti-EGFL7 antibody comprises the following light chain variable region sequence:
(SEQ ID NO: 21)
DIQMTQSPSSLSASVGDRVTITCRTSQSLVHINAITYLHWYQQKPGK
APKLLIYRVSNRFSGVPSRFSGSGSGTDFTLTISSLQPEDFATYYCG
QSTHVPLTFGQGTKVEIKR.


SEQUENCE LISTING (21 sequences)

SEQ ID NO: 1 - protein, 15 aa, Artificial Sequence
  Description of Artificial Sequence Synthetic peptide
KASQSVDYSGDSYMS

SEQ ID NO: 2 - protein, 7 aa, Artificial Sequence
  Description of Artificial Sequence Synthetic peptide
GASYRES

SEQ ID NO: 3 - protein, 9 aa, Artificial Sequence
  Description of Artificial Sequence Synthetic peptide
QQNNEEPYT

SEQ ID NO: 4 - protein, 10 aa, Artificial Sequence
  Description of Artificial Sequence Synthetic peptide
GHTFTTYGMS

SEQ ID NO: 5 - protein, 18 aa, Artificial Sequence
  Description of Artificial Sequence Synthetic peptide
GWINTHSGVPTYADDFKG

SEQ ID NO: 6 - protein, 8 aa, Artificial Sequence
  Description of Artificial Sequence Synthetic peptide
LGSYAVDY

SEQ ID NO: 7 - protein, 117 aa, Artificial Sequence
  Description of Artificial Sequence Synthetic polypeptide
EVQLVESGGGLVQPGGSLRLSCAASGHTFTTYGMSWVRQAPGKGLEWVGWINTHSGVPTYADDFKGRFTISLDNSKNTAYLQMNSLRAEDTAVYYCARLGSYAVDYWGQGTLVTVSS

SEQ ID NO: 8 - protein, 117 aa, Artificial Sequence
  Description of Artificial Sequence Synthetic polypeptide
EIQLVESGGGLVQPGGSLRLSCAASGHTFTTYGMSWVRQAPGKGLEWMGWINTHSGVPTYADDFKGRFTISLDNSKSTAYLQMNSLRAEDTAVYFCARLGSYAVDYWGQGTLVTVSS

SEQ ID NO: 9 - protein, 112 aa, Artificial Sequence
  Description of Artificial Sequence Synthetic polypeptide
DIQMTQSPSSLSASVGDRVTITCKASQSVDYSGDSYMSWYQQKPGKAPKLLIYGASYRESGVPSRFSGSGSGTDFTLTISSLQPEDFATYYCQQNNEEPYTFGQGTKVEIKR

SEQ ID NO: 10 - protein, 16 aa, Artificial Sequence
  Description of Artificial Sequence Synthetic peptide
RTSQSLVHINGITYLH

SEQ ID NO: 11 - protein, 7 aa, Artificial Sequence
  Description of Artificial Sequence Synthetic peptide
RVSNRFS

SEQ ID NO: 12 - protein, 9 aa, Artificial Sequence
  Description of Artificial Sequence Synthetic peptide
GQSTHVPLT

SEQ ID NO: 13 - protein, 10 aa, Artificial Sequence
  Description of Artificial Sequence Synthetic peptide
GYTFIDYYMN

SEQ ID NO: 14 - protein, 18 aa, Artificial Sequence
  Description of Artificial Sequence Synthetic peptide
GDINLDNGGTHYNQKFKG

SEQ ID NO: 15 - protein, 16 aa, Artificial Sequence
  Description of Artificial Sequence Synthetic peptide
AREGVYHDYDDYAMDY

SEQ ID NO: 16 - protein, 123 aa, Artificial Sequence
  Description of Artificial Sequence Synthetic polypeptide
EVQLVESGGGLVQPGGSLRLSCAASGYTFIDYYMNWVRQAPGKGLEWVGDINLDNGGTHYNQKFKGRFTISRDKSKNTAYLQMNSLRAEDTAVYYCAREGVYHDYDDYAMDYWGQGTLVTVSS

SEQ ID NO: 17 - protein, 113 aa, Artificial Sequence
  Description of Artificial Sequence Synthetic polypeptide
DIQMTQSPSSLSASVGDRVTITCRTSQSLVHINGITYLHWYQQKPGKAPKLLIYRVSNRFSGVPSRFSGSGSGTDFTLTISSLQPEDFATYYCGQSTHVPLTFGQGTKVEIKR

SEQ ID NO: 18 - protein, 16 aa, Artificial Sequence
  Description of Artificial Sequence Synthetic peptide
RTSQSLVHINAITYLH

SEQ ID NO: 19 - protein, 18 aa, Artificial Sequence
  Description of Artificial Sequence Synthetic peptide
GDINLDNSGTHYNQKFKG

SEQ ID NO: 20 - protein, 123 aa, Artificial Sequence [sequence omitted]

SEQ ID NO: 21 - protein, 113 aa, Artificial Sequence
  Description of Artificial Sequence Synthetic polypeptide
DIQMTQSPSSLSASVGDRVTITCRTSQSLVHINAITYLHWYQQKPGKAPKLLIYRVSNRFSGVPSRFSGSGSGTDFTLTISSLQPEDFATYYCGQSTHVPLTFGQGTKVEIKR